(19) United States
(12) Patent Application Publication
(10) Pub. No.: US 2024/0285563 A1
(43) Pub. Date: Aug. 29, 2024

(54) USE OF (S)-3-AMINO-4-(DIFLUOROMETHYLENYL) CYCLOPENT-1-ENE-1-CARBOXYLIC ACID IN THE TREATMENT OF RHEUMATOID ARTHRITIS

(71) Applicant: Ovid Therapeutics Inc., New York, NY (US)

(72) Inventor: Patrick Sarmiere, New York, NY (US)

(21) Appl. No.: 18/443,578

(22) Filed: Feb. 16, 2024

Related U.S. Application Data
(60) Provisional application No. 63/485,614, filed on Feb. 17, 2023.

(51) Int. Cl. A61K 31/196

(52) U.S. Cl. CPC A61K 31/196

(57) ABSTRACT

Methods and compositions for treating rheumatoid arthritis are provided which include administering an effective amount of (S)-3-amino-4-(difluoromethylenyl)cyclopent-1-ene-1-carboxylic acid or a pharmaceutically acceptable salt thereof to a subject in need thereof. In embodiments, compositions for treating rheumatoid arthritis that include (S)-3-amino-4-(difluoromethylenyl)cyclopent-1-ene-1-carboxylic acid or a pharmaceutically acceptable salt thereof are administered to a subject in need thereof.

### CROSS REFERENCE TO RELATED APPLICATION

This application claims benefit of and priority to U.S. Provisional Application No. 63/485,614, filed on Feb. 17, 2023, and which is incorporated herein by reference in its entirety.

### TECHNICAL FIELD

Treatment of rheumatoid arthritis with (S)-3-amino-4-(difluoromethylenyl) cyclopent-1-ene-1-carboxylic acid or pharmaceutically acceptable salt thereof.

### BACKGROUND

According to the NIH National Library of Medicine, National Center For Biotechnology Information, StatPearls, Chauhan et al., (2022), and the NIH—National Institute of Arthritis and Musculoskeletal and Skin Diseases, rheumatoid arthritis (RA) is a systemic autoimmune disease characterized by inflammatory arthritis and extra-articular involvement. It is a chronic inflammatory disorder caused in many cases by the interaction between genes and environmental factors including tobacco that primarily involves synovial joints. Various immune cells cause inflammation in the inner lining of the joint, called the synovium.

RA typically starts in small peripheral joints, is usually symmetric, and progresses to involve proximal joints if left untreated. Joint inflammation over time leads to the destruction of the joint with loss of cartilage and bone erosions. This inflammation becomes chronic, and the synovium thickens due to an increase of cells, production of proteins, and other factors in the joint, which can lead to pain, redness, and warmth. As RA progresses, the thickened and inflamed synovium pushes further into the joint and destroys the cartilage and bone within the joint. As the joint capsule stretches, the forces cause changes within the joint structure.

Symptoms of RA include joint pain at rest and when moving, along with tenderness, swelling, and warmth of the joint; joint stiffness that lasts longer than 30 minutes, typically after waking in the morning or after resting for a long period of time; fatigue—feeling unusually tired or having low energy; occasional low-grade fever; and loss of appetite. RA can cause other medical problems, such as: rheumatoid nodules that are firm lumps just below the skin, typically on the hands and elbows; anemia due to low red blood cell counts; neck pain; dry eyes and mouth; inflammation of the blood vessels, the lung tissue, airways, the lining of the lungs, or the sac enclosing the heart; and lung disease, characterized by scarring and inflammation of the lungs that can be severe in some people with RA. RA with a symptom duration of fewer than six months is defined as early RA, and when the symptoms have been present for more than six months, it is defined as established RA.

Rheumatoid arthritis has no cure and is a progressive disease associated with morbidity and increased mortality. All individuals will experience multiple exacerbations, and without treatment tend to have poor outcomes with disability. The treatment of patients with rheumatoid arthritis requires both pharmacological and non-pharmacological therapy.

Disease-modifying antirheumatic drugs (DMARDs) typically used in treating RA include methotrexate, hydroxychloroquine, sulfasalazine, and leflunomide. Anti-TNF-alpha inhibitors include etanercept, infliximab, adalimumab, golimumab, and certolizumab pegol. Other biologic DMARDs include interleukin (IL) 6 inhibitors such as tocilizumab and sarilumab, T-cell costimulation inhibitors such as abatacept (CTLA4-Ig), and the anti-CD20 B-cell depleting monoclonal antibody such as rituximab. Targeted synthetic DMARDs include Janus kinases (JAK) inhibitors such as tofacitinib, baricitinib, and upadacitinib.

Non-steroidal anti-inflammatory drugs (NSAIDs) do not have any disease-modifying effects but are commonly used to relieve symptoms related to joint inflammation and pain. There are about 20 such drugs (depending on country) that can be effective at full doses. There is some variation in side effects and toxicities. However, NSAIDs have the potential for gastrointestinal, renal, and hematologic toxicity.

Corticosteroids are used commonly in patients with RA. Short courses of corticosteroids can be used for mild flares of RA. Intra-articular corticosteroids can be used for single-joint flares. About 50% of patients with RA require low-dose corticosteroids (e.g., prednisone 2.5-7.5 mg daily to maintain control of their disease). Long-term corticosteroids are associated with numerous toxicities including weight gain, osteoporosis, and increased risk of infections.

Nonbiologic DMARDs include methotrexate, hydroxychloroquine (HCQ), azathioprine (AZA), sulfasalazine, leflunomide, and cyclosporine. Triple therapy consists of the combination of methotrexate, hydroxychloroquine, and sulfasalazine has been shown to be an effective regimen for RA. However, triple therapy is not well tolerated.

Tumor necrosis factor (TNF) inhibitors include etanercept, infliximab, adalimumab, certolizumab, and golimumab. The most concerning adverse effect of these agents is opportunistic infections and reactivation of latent tuberculosis.

Vigabatrin is a gamma aminobutyric acid aminotransferase (GABA-AT) inhibitor that has been used to treat treatment resistant epilepsy and infantile spasms. Vigabatrin has been associated with some potentially serious side effects. Its use has been limited due to potential retinal toxicity and subsequent visual field defects. (1S,3S)-3-amino+difluoromethylenyl-1-cyclopentanoic acid (also known as CPP-115) is a GABA-AT inhibitor which is 186 times more efficient in inactivating GABA-AT than vigabatrin. Preclinical data for CPP-115 has been reported to show that significantly lower drug dosages afford comparable pharmacokinetics, improved tolerability, and a more favorable toxicity profile when compared with vigabatrin. Sec, Prescot et al., Neuropsychopharmacology (2018) 43, 646-654. See also, U.S. Pat. No. 9,993,449, incorporated herein by reference. (S)-3-amino-4-(difluoromethylenyl)cyclopent-1-ene-1-carboxylic acid (also known as OV329) is a GABA-AT inhibitor that has been shown to be 9.8 times more efficient as an inactivator of GABA-AT than CPP-115. Id.

There remains a need for additional pharmaceutical therapies to treat RA that is effective while offering reduced side effects as compared to existing pharmaceutical therapies.

### SUMMARY

Methods and compositions for treating rheumatoid arthritis are provided. In embodiments, methods for treating rheumatoid arthritis include administering an effective amount of (S)-3-amino-4-(difluoromethylenyl)cyclopent-1-ene-1-carboxylic acid or a pharmaceutically acceptable salt thereof to a subject in need thereof. In embodiments, compositions for treating rheumatoid arthritis which include (S)-3-amino-4-(difluoromethylenyl)cyclopent-1-ene-1-carboxylic acid or a pharmaceutically acceptable salt thereof are administered to a subject in need thereof. In embodiments, methods of treating rheumatoid arthritis include administering (S)-3-amino-4-(difluoromethylenyl)cyclopent-1-ene-1-carboxylic acid or a pharmaceutically acceptable salt thereof to a subject in need thereof to provide improvement in one or more symptoms of rheumatoid arthritis in the subject. In embodiments, methods of treating rheumatoid arthritis include administering (S)-3-amino-4-(difluoromethylenyl)cyclopent-1-ene-1-carboxylic acid or a pharmaceutically acceptable salt thereof to a subject in need thereof to provide improvement in symptoms of rheumatoid arthritis in the subject the next day after administering (S)-3-amino-4-(difluoromethylenyl)cyclopent-1-ene-1-carboxylic acid or a pharmaceutically acceptable salt thereof. In embodiments, an effective amount of (S)-3-amino-4-(difluoromethylenyl)cyclopent-1-ene-1-carboxylic acid or a pharmaceutically acceptable salt thereof is between about 0.01 mg to about 750 mg. In embodiments, an effective amount of (S)-3-amino-4-(difluoromethylenyl)cyclopent-1-ene-1-carboxylic acid or a pharmaceutically acceptable salt thereof is administered to a subject once, twice, three times or four times daily. In embodiments, the rheumatoid arthritis being treated is early rheumatoid arthritis. In embodiments, the rheumatoid arthritis being treated is established rheumatoid arthritis.

### DETAILED DESCRIPTION

Methods and compositions for treating rheumatoid arthritis (RA) are provided. In accordance with the present disclosure methods and compositions for treating rheumatoid arthritis are provided that relieve symptoms of rheumatoid arthritis without certain unwanted side effects associated with commonly prescribed medications for rheumatoid arthritis or other GABA-AT inhibitors. In embodiments, methods for treating rheumatoid arthritis include administering an effective amount of (S)-3-amino-4-(difluoromethylenyl)cyclopent-1-ene-1-carboxylic acid or a pharmaceutically acceptable salt thereof to a subject in need thereof. In embodiments, compositions for treating rheumatoid arthritis which include (S)-3-amino-4-(difluoromethylenyl)cyclopent-1-ene-1-carboxylic acid or a pharmaceutically acceptable salt thereof are administered to a subject in need thereof. In embodiments, methods of treating rheumatoid arthritis include administering (S)-3-amino-4-(difluoromethylenyl)cyclopent-1-ene-1-carboxylic acid or a pharmaceutically acceptable salt thereof to a subject in need thereof to provide improvement in one or more symptoms of rheumatoid arthritis in the subject. In embodiments, methods of treating rheumatoid arthritis include administering (S)-3-amino-4-(difluoromethylenyl)cyclopent-1-ene-1-carboxylic acid or a pharmaceutically acceptable salt thereof to a subject in need thereof to provide improvement in symptoms of rheumatoid arthritis in the subject the next day after administering (S)-3-amino-4-(difluoromethylenyl)cyclopent-1-ene-1-carboxylic acid or a pharmaceutically acceptable salt thereof. In embodiments, an effective amount of (S)-3-amino-4-(difluoromethylenyl)cyclopent-1-ene-1-carboxylic acid or a pharmaceutically acceptable salt thereof is between about 0.01 mg to about 750 mg.

In embodiments, the rheumatoid arthritis being treated is early rheumatoid arthritis. In embodiments, the rheumatoid arthritis being treated is established rheumatoid arthritis.

The structure of (S)-3-amino-4-(difluoromethylenyl)cyclopent-1-ene-1-carboxylic acid may be represented as follows:

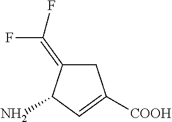 [SMILES: N[C@H]1C=C(C(=O)O)CC1=C(F)F]

In embodiments, (S)-3-amino-4-(difluoromethylenyl)cyclopent-1-ene-1-carboxylic acid may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, pantothenic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, hydroiodic, sulfuric, sulfamic, phosphoric or nitric acid addition salts may be used.

The pathogenesis of rheumatoid arthritis begins with an inflammatory cascade. Autoimmunity in rheumatoid arthritis starts at the molecular and cellular level. Rheumatoid Factor (RF) and anti-citrullinated protein antibodies (ACPAs) are autoantibodies, i.e., antibodies to an individual's own proteins, that are associated with rheumatoid arthritis. RF and ACPAs are present in the majority of patients with rheumatoid arthritis. With time the concentration of ACPA and serum cytokine levels increase. Some patients eventually transition from autoimmunity to immune-mediated inflammation primarily focused in the synovium. These autoantibodies are produced by plasma cells in the synovium. The synovium in RA is infiltrated by immune cells, which include innate immune cells (monocytes, dendritic cells, mast cells) and adaptive immune cells (T-helper 1, Th1); T-helper 17, Th17), B cells, and plasma cells. Synovial fibroblast-like synovial cells (FSC) are activated. Neutrophils are not present in the synovium but egress from the blood to the synovial fluid. Cytokines and chemokines such as tumor necrosis factor (TNF), interleukin-6 (IL-6), and granulocyte-monocyte colony-stimulating factor (GM-CSF) activate endothelial cells and attract immune cells within the synovial compartment. The FSC in the rheumatoid synovium changes to an invasive phenotype. FSC and inflammatory cells produce receptor activator of nuclear factor κ B ligand (RANKL) which leads to osteoclast generation resulting in bone erosions, a hallmark feature of rheumatoid arthritis.

Gamma-amino butyric acid (GABA) is the chief inhibitory neurotransmitter in the mature mammalian CNS. Outside the brain, GABA is produced by pancreatic B-cells as well as T cells and macrophages that express all components of GABAergic system, including its receptors, transporters, and metabolic enzymes. GABA acts as a negative regulator of macrophage production of inflammatory cytokines and T cell activation by blocking calcium signaling and NFκB activity.

Many different types of murine and human immune cells express various GABA receptors (GABA-Rs). Sec, Tian et al., Sci Rep 11, 5402 (2021). https://doi.org/10.1038/s41598-021-84751-3. GABA has been shown to limit murine T cell production of IL-21, IFNγ2-4, TNFα3, and IL-123 while promoting TGFß and Treg responses. Id. Antigen presenting cells (APCs) such as macrophages also express GABA_{A}-Rs and their activation inhibits their inflammatory activity. Id. For example, GABA or a GABA_{A}-R agonist reduced the secretion of IL-6, IL-1ß, IL-12 and/or TNFα from LPS-stimulated murine macrophages. Id.

(S)-3-amino-4-(difluoromethylenyl)cyclopent-1-ene-1-carboxylic acid is a gamma-aminobutyric acid aminotransferase (GABA-AT) inhibitor. GABA-AT is a pyridoxal 5′-phosphate-dependent enzyme responsible for the degradation of the inhibitory neurotransmitter GABA. (S)-3-amino-4-(difluoromethylenyl)cyclopent-1-ene-1-carboxylic acid has a higher binding affinity to GABA-AT as compared to CPP-115 (K_{I} values of 1 and CPP-115 have been found to be 9.7 UM and 59 μM, respectively), and (S)-3-amino-4-(difluoromethylenyl)cyclopent-1-ene-1-carboxylic acid inactivates GABA-AT at a greater rate than CPP-115 (K_{inact} values of (S)-3-amino-4-(difluoromethylenyl)cyclopent-1-ene-1-carboxylic acid and CPP-115 were 3.32 min 1 and 2.05 mm 1, respectively). Sec, U.S. Pat. No. 9,993,449. Overall, the efficiency constant for (S)-3-amino-4-(difluoromethylenyl)cyclopent-1-ene-1-carboxylic acid (k_{inact}/K_{I}=342 mM⁻¹min⁻¹) is 9.8 times larger than that for CPP-115 (K_{inact}/K_{I}=34.9 mM⁻¹ min⁻¹); therefore, (S)-3-amino-4-(difluoromethylenyl)cyclopent-1-ene-1-carboxylic acid is 9.8 times more efficient as an inactivator of GABA-AT than CPP-115. Unlike vigabatrin. CPP-115 was reported not to inactivate or inhibit off-target enzymes, such as aspartate aminotransferase (Asp-AT) and alanine aminotransferase (Ala-AT), which could have contributed to its larger margin of safety than vigabatrin. (S)-3-amino-4-(difluoromethylenyl)cyclopent-1-ene-1-carboxylic acid is a very weak reversible inhibitor of both Asp-AT and Ala-AT with an IC₅₀>4 mM. CPP-115 is a moderate inactivator of OAT with a K; value of 0.116 mM and a kina value of 0.097 min 1. (S)-3-amino-4-(difluoromethylenyl)cyclopent-1-ene-1-carboxylic acid is a potent inactivator of OAT with a K_{I} value of 0.0033 mM and a K_{inact} value of 0.025 min⁻¹. By comparison of the k_{inact}/K_{I} value of (S)-3-amino-4-(difluoromethylenyl)cyclopent-1-ene-1-carboxylic acid (7.6 mM⁻¹min⁻¹) with that of CPP-115 (0.84 mM⁻¹min⁻¹), (S)-3-amino-4-(difluoromethylenyl)cyclopent-1-ene-1-carboxylic acid is 9.0 times more efficient an inactivator of OAT than CPP. 115, consistent with its higher efficiency as an inactivator of GABA-AT.

Without wishing to be bound by any particular theory, selective inhibition of GABA-AT by (S)-3-amino-4-(difluoromethylenyl)cyclopent-1-ene-1-carboxylic acid or a pharmaceutically acceptable salt thereof, raises GABA concentrations where GABA is present. By increasing GABA concentrations GABA_{A}-Rs are activated which inhibits the synovium damaging inflammatory activity of lymphocytes and macrophages while also reducing production of inflammatory cytokines, thus inhibiting the disease process.

Rheumatoid arthritis symptoms which can be reduced or alleviated by administration of (S)-3-amino-4-(difluoromethylenyl)cyclopent-1-ene-1-carboxylic acid or a pharmaceutically acceptable salt thereof include joint pain at rest and when moving, along with tenderness, swelling, and warmth of the joint; joint stiffness that lasts longer than 30 minutes, typically after waking in the morning or after resting for a long period of time; fatigue-feeling unusually tired or having low energy; occasional low-grade fever; and loss of appetite; formation of rheumatoid nodules; anemia due to low red blood cell counts; neck pain; dry eyes and mouth; inflammation of the blood vessels, lung tissue, airways, the lining of the lungs, or the sac enclosing the heart; and lung disease characterized by scarring and inflammation of the lungs.

In embodiments, the terms “effective amount” or “therapeutically effective amount” may be used interchangeably and refer to an amount of a compound, material, composition, medicament, or other material that is effective to achieve reduction, elimination or prophylaxis of rheumatoid arthritis with a minimum of side effects normally associated with RA drugs or other GABA-AT inhibitors. In embodiments, the “effective amount” is administered to a subject in need thereof wherein the rheumatoid arthritis being treated is early rheumatoid arthritis. In embodiments, the “effective amount” is administered to a subject in need thereof wherein the rheumatoid arthritis being treated is established rheumatoid arthritis.

In embodiments, an “effective amount” herein can range from about 0.01 mg to about 750 mg of (S)-3-amino-4-(difluoromethylenyl)cyclopent-1-ene-1-carboxylic acid or a pharmaceutically acceptable salt thereof given from one to four times or more a day. For example, a pharmaceutical composition including an effective amount of (S)-3-amino-4-(difluoromethylenyl)cyclopent-1-ene-1-carboxylic acid or a pharmaceutically acceptable salt thereof may contain from about 0.01 mg to about 0.1 mg, about 0.1 mg to about 1 mg, 1 mg to about 5 mg, about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, about 45 mg to about 50 mg, about 50 mg to about 55 mg, about 55 mg to about 60 mg, about 60 mg to about 65 mg, about 65 mg to about 70 mg, about 70 mg to about 75 mg, about 75 mg to about 80 mg, about 80 mg to about 85 mg, about 85 mg to about 90 mg, about 90 mg to about 95 mg, about 95 mg to about 100 mg, about 100 mg to about 105 mg, about 105 mg to about 110 mg, about 110 mg to about 115 mg, about 115 mg to about 120 mg, about 120 mg to about 125 mg, about 125 mg to about 130 mg, about 130 mg to about 135 mg, about 135 mg to about 140 mg, about 140 mg to about 145 mg, about 145 mg to about 150, about 150 mg to about 155 mg, about 155 mg to about 160 mg, about 160 mg to about 165 mg, about 165 mg to about 170 mg, about 170 mg to about 175 mg, about 175 mg to about 180 mg, about 180 mg to about 185 mg, about 185 mg to about 190 mg, about 190 mg to about 195 mg, about 195 mg to about 200 mg, about 200 mg to about 205 mg, about 205 mg to about 210 mg, about 210 mg to about 215 mg, about 215 mg to about 220 mg, about 220 mg to about 225 mg, about 225 mg to about 230 mg, about 230 mg to about 235 mg, about 235 mg to about 240 mg, about 240 mg to about 245 mg, about 245 mg to about 250, about 250 mg to about 255 mg, about 255 mg to about 260 mg, about 260 mg to about 265 mg, about 265 mg to about 270 mg, about 270 mg to about 275 mg, about 275 mg to about 280 mg, about 280 mg to about 285 mg, about 285 mg to about 290 mg, about 290 mg to about 295 mg, about 295 mg to about 300 mg, about 300 mg to about 305 mg, about 305 mg to about 310 mg, about 310 mg to about 315 mg, about 315 mg to about 320 mg, about 320 mg to about 325 mg, about 325 mg to about 330 mg, 330 mg to about 335 mg, about 335 mg to about 340 mg, about 340 mg to about 345 mg, about 345 mg to about 350, about 350 mg to about 355 mg, about 355 mg to about 360 mg, about 360 mg to about 365 mg, about 365 mg to about 370 mg, about 370 mg to about 375 mg, about 375 mg to about 380 mg, about 380 mg to about 385 mg, about 385 mg to about 390 mg, about 390 mg to about 395 mg, about 395 mg to about 400 mg, about 400 mg to about 405 mg, about 405 mg to about 410 mg, about 410 mg to about 415 mg, about 415 mg to about 420 mg, about 420 mg to about 425 mg, about 425 mg to about 430 mg, about 430 mg to about 435 mg, about 435 mg to about 440 mg, about 440 mg to about 445 mg, about 445 mg to about 450, about 450 mg to about 455 mg, about 455 mg to about 460 mg, about 460 mg to about 465 mg, about 465 mg to about 470 mg, about 470 mg to about 475 mg, about 475 mg to about 480 mg, about 480 mg to about 485 mg, about 485 mg to about 490 mg, about 490 mg to about 495 mg, or about 495 mg to about 500 mg of (S)-3-amino-4-(difluoromethylenyl)cyclopent-1-ene-1-carboxylic acid or a pharmaceutically acceptable salt thereof.

In embodiments a pharmaceutical composition containing an effective amount of (S)-3-amino-4-(difluoromethylenyl)cyclopent-1-ene-1-carboxylic acid or a pharmaceutically acceptable salt thereof includes 0.01 mg, 0.1 mg, 0.5 mg, 1 mg, 2 mg, 3 mg, 4 mg, 5 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 230 mg, 331 mg, 332 mg, 333 mg, 334 mg, 335 mg, 336 mg, 337 mg, 338 mg, 339 mg, 340 mg, 241 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 237 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, or 500 mg, of (S)-3-amino-4-(difluoromethylenyl)cyclopent-1-ene-1-carboxylic acid or a pharmaceutically acceptable salt thereof.

In embodiments, (S)-3-amino-4-(difluoromethylenyl)cyclopent-1-ene-1-carboxylic acid or a pharmaceutically acceptable salt thereof is administered to a subject at from about 0.01 mg/per day to about 750 mg/per day. For example, in embodiments, (S)-3-amino-4-(difluoromethylenyl)cyclopent-1-ene-1-carboxylic acid or a pharmaceutically acceptable salt thereof is administered to a subject at about 0.01 mg/per day, about 0.1 mg/per day, about 0.5 mg/per day about 1 mg/per day, about 5 mg/per day, about 10 mg/per day, about 15 mg/per day, about 20 mg/per day, about 25 mg/per day, about 30 mg/per day, about 35 mg/per day, about 40 mg/per day, about 45 mg/per day, about 50 mg/per day, about 60 mg/per day, about 65 mg/per day, about 70 mg/per day, about 75 mg/per day, about 80 mg/per day, about 85 mg/per day, about 90 mg/per day, about 95 mg/per day, about 100 mg/per day, about 105 mg/per day, about 110 mg/per day, about 115 mg/per day, about 120 mg/per day, about 125 mg/per day, about 130 mg/per day, about 135 mg/per day, about 140 mg/per day, about 145 mg/per day, about 150 mg/per day, about 155 mg/per day, about 160 mg/per day, about 165 mg/per day, about 170 mg/per day, about 175 mg/per day, about 180 mg/per day, about 185 mg/per day, about 190 mg/per day, about 195 mg/per day, about 200 mg/per day, about 205 mg/per day, about 210 mg/per day, about 215 mg/per day, about 220 mg/per day, about 225 mg/per day, about 230 mg/per day, about 235 mg/per day, about 240 mg/per day, about 245 mg/per day, about 250 mg/per day, about 255 mg/per day, about 260 mg/per day, about 265 mg/per day, about 270 mg/per day, about 275 mg/per day, about 280 mg/per day, about 285 mg/per day, about 290 mg/per day, about 295 mg/per day, about 300 mg/per day, about 305 mg/per day, about 310 mg/per day, about 315 mg/per day, about 320 mg/per day, about 325 mg/per day, about 330 mg/per day, about 335 mg/per day, about 340 mg/per day, about 345 mg/per day, about 350 mg/per day, about 355 mg/per day, about 360 mg/per day, about 365 mg/per day, about 370 mg/per day, about 375 mg/per day, about 380 mg/per day, about 385 mg/per day, about 390 mg/per day, about 395 mg/per day, about 400 mg/per day, about 405 mg/per day, about 410 mg/per day, about 415 mg/per day, about 420 mg/per day, about 425 mg/per day, about 430 mg/per day, about 435 mg/per day, about 440 mg/per day, about 445 mg/per day, about 450 mg/per day, about 455 mg/per day, about 460 mg/per day, about 465 mg/per day, about 470 mg/per day, about 475 mg/per day, about 480 mg/per day, about 485 mg/per day, about 490 mg/per day, about 495 mg/per day, about 500 mg/per day, about 505 mg/per day, about 510 mg/per day, about 515 mg/per day, about 520 mg/per day, about 525 mg/per day, about 530 mg/per day, about 535 mg/per day, about 540 mg/per day, about 545 mg/per day, about 550 mg/per day, about 555 mg/per day, about 560 mg/per day, about 565 mg/per day, about 570 mg/per day, about 575 mg/per day, about 580 mg/per day, about 585 mg/per day, about 590 mg/per day, about 595 mg/per day, about 600 mg/per day, about 605 mg/per day, about 610 mg/per day, about 615 mg/per day, about 620 mg/per day, about 625 mg/per day, about 630 mg/per day, about 635 mg/per day, about 640 mg/per day, about 645 mg/per day, about 650 mg/per day, about 655 mg/per day, about 660 mg/per day, about 665 mg/per day, about 670 mg/per day, about 675 mg/per day, about 680 mg/per day, about 685 mg/per day, about 690 mg/per day, about 695 mg/per day, about 700 mg/per day, about 705 mg/per day, about 710 mg/per day, about 715 mg/per day, about 720 mg/per day, about 725 mg/per day, about 730 mg/per day, about 735 mg/per day, about 740 mg/per day, about 745 mg/per day, or about 750 mg/per day, in one, two, three, four or more doses. In embodiments, the subject may be started at a low dose and the dosage is escalated over time.

In embodiments, (S)-3-amino-4-(difluoromethylenyl)cyclopent-1-ene-1-carboxylic acid or a pharmaceutically acceptable salt thereof is administered to a subject diagnosed with rheumatoid arthritis via a pharmaceutical composition. Pharmaceutical compositions herein encompass dosage forms. Dosage forms herein encompass unit doses. In embodiments, as discussed below, various dosage forms including conventional formulations and modified release formulations can be administered one, two, three, four or more times daily. In embodiments, (S)-3-amino-4-(difluoromethylenyl) cyclopent-1-ene-1-carboxylic acid or a pharmaceutically acceptable salt thereof is administered to a subject once or twice a day, (e.g., morning and/or evening). In embodiments, (S)-3-amino-4-(difluoromethylenyl) cyclopent-1-ene-1-carboxylic acid or a pharmaceutically acceptable salt thereof is administered to a subject three times a day, (e.g., morning, afternoon and at bedtime, or every 8 hours). In embodiments, (S)-3-amino-4-(difluoromethylenyl) cyclopent-1-ene-1-carboxylic acid or a pharmaceutically acceptable salt thereof is administered to a subject four times a day, (e.g., morning, afternoon, evening and at bedtime, or every 6 hours). In embodiments, (S)-3-amino-4-(difluoromethylenyl) cyclopent-1-ene-1-carboxylic acid or a pharmaceutically acceptable salt thereof is administered to a subject via continuous infusion. In embodiments, (S)-3-amino-4-(difluoromethylenyl) cyclopent-1-ene-1-carboxylic acid or a pharmaceutically acceptable salt thereof is administered to a subject at the start of an acute rheumatoid arthritis symptom episode, whenever that may occur. Any suitable route of administration may be utilized, e.g., oral, rectal, nasal, pulmonary, vaginal, sublingual, transdermal, intravenous, intraarterial, epidural, intramuscular, intraperitoneal and subcutaneous routes. Suitable dosage forms include tablets, capsules, oral liquids, powders, aerosols, transdermal modalities such as topical liquids, patches, creams and ointments, parenteral formulations and suppositories. In embodiments, (S)-3-amino-4-(difluoromethylenyl) cyclopent-1-ene-1-carboxylic acid or a pharmaceutically acceptable salt thereof is used to manufacture a medicament for treatment of rheumatoid arthritis. In embodiments, the rheumatoid arthritis being treated early rheumatoid arthritis. In embodiments, the rheumatoid arthritis being treated established rheumatoid arthritis.

In embodiments, methods of treating rheumatoid arthritis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of rheumatoid arthritis for more than 1 hour after administration to the subject. In embodiments, methods of treating rheumatoid arthritis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of rheumatoid arthritis for more than 2 hours after administration to the subject. In embodiments, methods of treating rheumatoid arthritis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of rheumatoid arthritis for more than 3 hours after administration to the subject. In embodiments, methods of treating rheumatoid arthritis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of rheumatoid arthritis for more than 4 hours after administration to the subject. In embodiments, methods of treating rheumatoid arthritis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of rheumatoid arthritis for more than 6 hours after administration to the subject. In embodiments, methods of treating rheumatoid arthritis are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of rheumatoid arthritis for more than 8, 10, 12, 14, 16, 18, 20, 22 or 24 hours after administration to the subject. In embodiments, the pharmaceutical compositions provide improvement of next day functioning of the subject diagnosed with rheumatoid arthritis. For example, the pharmaceutical compositions may provide improvement in symptoms of rheumatoid arthritis for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

In embodiments, as mentioned previously, pharmaceutical compositions herein may be provided with conventional release or modified release profiles. Pharmaceutical compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers.

In embodiments, pharmaceutical compositions herein are modified release dosage forms which provide modified release profiles. Modified release profiles may exhibit immediate release, delayed release, or extended release profiles. Conventional (or unmodified) release oral dosage forms such as tablets, capsules, suppositories, syrups, solutions and suspensions typically release medications into the mouth, stomach or intestines as the tablet, capsule shell or suppository dissolves, or, in the case of syrups, solutions and suspensions, when they are swallowed. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Subjects with rheumatoid arthritis may exhibit such behavior. ODDF's can provide rapid delivery of medication to the blood stream through mucosa resulting in a rapid onset of action. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which (S)-3-amino-4-(difluoromethylenyl)cyclopent-1-ene-1-carboxylic acid or a pharmaceutically acceptable salt thereof is applied to beads, e.g., confectioners nonpareil beads, and then coated with conventional release retarding materials such as waxes, enteric coatings and the like. In embodiments, beads can be formed in which (S)-3-amino-4-(difluoromethylenyl)cyclopent-1-ene-1-carboxylic acid or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as (S)-3-amino-4-(difluoromethylenyl)cyclopent-1-ene-1-carboxylic acid or a pharmaceutically acceptable salt thereof at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles is covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles is covered with a coating which delays release of the drug.

Delayed release dosage forms are known to those skilled in the art. For example, coated delayed release beads or granules in which (S)-3-amino-4-(difluoromethylenyl)cyclopent-1-ene-1-carboxylic acid or a pharmaceutically acceptable salt thereof is applied to beads, e.g., confectioners nonpareil beads, and then coated with conventional release delaying materials such as waxes, enteric coatings and the like. In embodiments, beads can be formed in which (S)-3-amino-4-(difluoromethylenyl)cyclopent-1-ene-1-carboxylic acid or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of (S)-3-amino-4-(difluoromethylenyl)cyclopent-1-ene-1-carboxylic acid or a pharmaceutically acceptable salt thereof can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, (S)-3-amino-4-(difluoromethylenyl)cyclopent-1-ene-1-carboxylic acid or a pharmaceutically acceptable salt thereof is incorporated into porous inert carriers that provide delayed release profiles. In embodiments, the porous inert carriers incorporate channels or passages from which the drug diffuses into surrounding fluids. In embodiments, (S)-3-amino-4-(difluoromethylenyl)cyclopent-1-ene-1-carboxylic acid or a pharmaceutically acceptable salt thereof is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, pharmaceutical compositions described herein are suitable for parenteral administration, including, e.g., intramuscular (i.m.), intravenous (i.v.), subcutaneous (s.c.), intraperitoneal (i.p.), epidural, or intrathecal (i.t.). Parenteral compositions should be sterile for administration by injection, infusion or implantation into the body and may be packaged in either single-dose or multi-dose containers. In embodiments, liquid pharmaceutical compositions for parenteral administration to a subject include an active substance, e.g., (S)-3-amino-4-(difluoromethylenyl)cyclopent-1-ene-1-carboxylic acid or a pharmaceutically acceptable salt thereof in any of the respective amounts described above. In embodiments, the pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are contained in a bag, a glass vial, a plastic vial, or a bottle.

Pharmaceutical compositions for parenteral administration provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of (S)-3-amino-4-(difluoromethylenyl)cyclopent-1-ene-1-carboxylic acid or a pharmaceutically acceptable salt thereof used in the composition. Thus, parenteral compositions are provided wherein there is no incompatibility between any of the components of the dosage form.

In embodiments, parenteral compositions (S)-3-amino-4-difluoromethylenyl)cyclopent-1-ene-1-carboxylic acid or a pharmaceutically acceptable salt thereof include a stabilizing amount of at least one excipient. For example, excipients may be selected from the group consisting of buffering agents, solubilizing agents, tonicity agents, antioxidants, chelating agents, antimicrobial agents, and preservative. One skilled in the art will appreciate that an excipient may have more than one function and be classified in one or more defined group.

In embodiments, parenteral compositions (S)-3-amino-4-difluoromethylenyl)cyclopent-1-ene-1-carboxylic acid or a pharmaceutically acceptable salt thereof and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions of an active substance, e.g., (S)-3-amino-4-(difluoromethylenyl)cyclopent-1-ene-1-carboxylic acid or a pharmaceutically acceptable salt thereof are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

It should be understood that the dosage amounts of (S)-3-amino-4-(difluoromethylenyl)cyclopent-1-ene-1-carboxylic acid, or a pharmaceutically acceptable salt thereof that are provided herein are applicable to all the dosage forms described herein including conventional dosage forms, modified dosage forms, as well as the parenteral formulations described herein. Those skilled in the art will determine appropriate amounts depending on criteria such as dosage form, route of administration, subject tolerance, efficacy, therapeutic goal and therapeutic benefit, among other pharmaceutically acceptable criteria.

Clinical efficacy of treatment can be monitored using any method known in the art. Measurable parameters to monitor efficacy will depend on the condition being treated. For monitoring the status or improvement of rheumatoid arthritis, both subjective parameters (e.g., patient reporting) and objective parameters (e.g., f-MRI, allodynia, hyperalgesia, physical exam, gait, mobility, walking distance, nerve conduction velocity, electrophysiology, etc.) can be used.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value.

“Improvement” refers to the treatment of rheumatoid arthritis including all symptoms that are normally associated with rheumatoid arthritis.

“Improvement in next day functioning” or “wherein there is improvement in next day functioning” refers to improvement after waking from an overnight sleep period wherein the beneficial effect of administration of (S)-3-amino-4-(difluoromethylenyl)cyclopent-1-ene-1-carboxylic acid or a pharmaceutically acceptable salt thereof applies to symptoms of rheumatoid arthritis and is discernable, either subjectively by a subject or objectively by an observer, for a period of time, e.g., 2 hours, 3 hours, 4 hours, 5 hours, 6 hours, 12 hours, 24 hours, etc. after waking.

“Treating”, “treatment” or “treat” can refer to the following: reducing, improving, relieving, ameliorating, mitigating, inhibiting, reversing and/or alleviating rheumatoid arthritis in a subject, or delaying the appearance of symptoms of rheumatoid arthritis (prophylaxis) in a subject. In embodiments, “treating”, “treat” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating”, “treat” or “treatment” also refers to inhibiting or relieving rheumatoid arthritis, e.g., causing regression of rheumatoid arthritis or at least one of its clinical or subclinical symptoms. The benefit to a subject to be treated may be statistically significant, mathematically significant, or at least perceptible to the subject and/or the physician. Nonetheless, prophylactic (preventive) and therapeutic (curative) treatment are two separate embodiments of the disclosure herein.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Co-administered with”, “administered in combination with”, “a combination of” or “administered along with” may be used interchangeably and mean that two or more agents are administered in the course of therapy. The agents may be administered together at the same time or separately in spaced apart intervals. The agents may be administered in a single dosage form or in separate dosage forms.

“Subject in need thereof” includes individuals that have been diagnosed with rheumatoid arthritis. The methods and compositions including (S)-3-amino-4-(difluoromethylenyl)cyclopent-1-ene-1-carboxylic acid or a pharmaceutically acceptable salt thereof may be provided to any individual including, e.g., wherein the subject is a neonate, infant, a pediatric subject (6 months to 12 years), an adolescent subject (age 12-18 years) or an adult (over 18 years). Subjects include mammals. “Patient” and “subject” may be used interchangeably herein.

The term “pharmaceutically acceptable salt”, as used herein, refers to derivatives of the compounds defined herein, wherein the parent compound is modified by making acid or base salts thereof. Examples of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include but are not limited to those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts. The pharmaceutically acceptable salts can be synthesized from the parent compound, which contains a basic or acidic moiety, by conventional chemical methods.

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

What is claimed is:

1. A method of treating rheumatoid arthritis comprising administering (S)-3-amino-4-(difluoromethylenyl)cyclopent-1-ene-1-carboxylic acid or a pharmaceutically acceptable salt thereof to a subject in need thereof in an amount of from about 0.01 mg to about 750 mg.

2. The method of claim 1, wherein the subject is administered from 0.1 mg to 600 mg of (S)-3-amino-4-(difluoromethylenyl)cyclopent-1-ene-1-carboxylic acid or a pharmaceutically acceptable salt thereof.

3. The method of claim 1, wherein the subject is administered from 1 mg to 500 mg of (S)-3-amino-4-(difluoromethylenyl)cyclopent-1-ene-1-carboxylic acid or a pharmaceutically acceptable salt thereof.

4. The method of claim 1, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 400 mg.

5. The method of claim 1, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 300 mg.

6. The method of claim 1, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 200 mg.

7. The method of claim 1, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 100 mg.

8. The method of claim 1, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 50 mg.

9. The method of claim 1, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 25 mg.

10. The method of claim 1, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 10 mg.

11. The method of claim 1, wherein (S)-3-amino-4-(difluoromethylenyl)cyclopent-1-ene-1-carboxylic acid or a pharmaceutically acceptable salt thereof is administered from one to four times a day.

12. The method of claim 1, wherein administering is accomplished via a route selected from the group consisting of oral, buccal, sublingual, rectal, topical, intranasal, ophthalmic, vaginal and parenteral.

13. The method of claim 1, wherein treating rheumatoid arthritis ameliorates one or more symptoms of rheumatoid arthritis.

14. The method of claim 1, wherein the rheumatoid arthritis is early rheumatoid arthritis.

15. The method of claim 1, wherein the rheumatoid arthritis is established rheumatoid arthritis.
